(19) United States
(12) Patent Application Publication
(10) Pub. No.: US 2026/0361695 A1
(43) Pub. Date: Nov. 12, 2026

(54) AUTHENTICABLE DATA TRANSMISSION BY A MEDICAL DEVICE

(71) Applicant: Medtronic, Inc., Minneapolis, MN (US)

(72) Inventors: Bo Zhang, Shoreview, MN (US); Thomas J. August, Golden Valley, MN (US); Christopher T. House, Rosemount, MN (US); John Louis Clark, Champlin, MN (US); Robert D. Musto, Champlin, MN (US); John C. Stroebel, Blaine, MN (US); Joel B. Artmann, Elk River, MN (US)

(21) Appl. No.: 19/125,133

(22) PCT Filed: Oct. 25, 2023
(86) PCT No.: PCT/US2023/077796

§ 371 Date: Apr. 28, 2025

Related U.S. Application Data
(60) Provisional application No. 63/381,491, filed on Oct. 28, 2022.

(51) Int. Cl. H04L 9/40; G16H 40/67; H04L 9/32

(52) U.S. Cl. CPC H04L 63/0428; G16H 40/67; H04L 9/3247

(57) ABSTRACT

This disclosure is directed to devices, systems, and techniques for an implantable medical device (HMD) to transmit data that can be authenticated by computing devices that receive the data. The HMD may generate one or more data packets. The HMD may sign the one or more data packets using a private key to generate a digital signature associated with the one or more data packets. The IMD may send the one or more data packets and the digital signature to a computing device.

This application is an international application with provisional priority of U.S. Provisional Patent Application No. 63/381,491, filed 28 Oct. 2022, the entire contents of which is incorporated herein by reference.

### TECHNICAL FIELD

The disclosure relates to device communication between two or more devices.

### BACKGROUND

A computing device may be configured to receive communications from a medical device, such as a wearable medical device, an implantable medical device (IMD), or a partially implantable medical device. The medical device may be configured to monitor one or more physiological parameters of a patient and/or deliver therapy to suppress one or more symptoms of the patient. In the case of an IMD, for example, the IMD may be configured to sense and monitor a cardiac rhythm of the patient, be configured to deliver cardiac pacing or another electrical therapy to the patient, and/or be configured to terminate tachyarrhythmia by delivery of high energy shocks. Such IMDs include insertable cardiac monitors, pacemakers, implantable cardioverter-defibrillators (ICDs), cardiac resynchronization therapy (CRT) devices, pulmonary artery pressure sensors, and the like. Other types of medical devices include wearable cardiac monitors, wearable cardiac defibrillators, drug pumps, neurostimulators, and many other types of wearable, implantable or partially implantable medical devices.

A clinician or patient may use an external device to retrieve information collected by the IMD (or other medical device) and/or to configure or adjust one or more parameters of the monitoring and/or therapy provided by the IMD (or other medical device). Typically, the external device connects to the IMD via a wireless connection. In some examples, a wireless connection is established between the external device and the IMD using a Bluetooth Low Energy (BLE) wireless protocol.

### SUMMARY

In general, the disclosure is directed to devices, systems, and techniques for an medical device, such as an implantable, partially-implantable or wearable medical device, to transmit data to a computing device that can be authenticated by the computing device to verify the identity of the IMD that transmitted the data and to verify the integrity of the data transmitted by the IMD. Specifically, the IMD may store a private key and an associated digital certificate in memory, and the IMD may sign one or more data packets to be transmitted to a computing device using the private key to generate a digital signature associated with the one or more data packets. The IMD may transmit the one or more data packets and the associated digital signature along with the digital certificate to a computing device.

A computing device that receives the one or more data packets and the associated digital signature from the IMD may use the digital certificate to verify the identity of the IMD that transmitted the one or more data packets and to verify the integrity and authenticity of the one or more data packets received by the computing device. In particular, the computing device may store a public key that is part of an asymmetric key pair with the private key used by the IMD to sign the one or more data packets. As such, the computing device may use the public key to verify the identity of the IMD that transmitted the one or more data packets and to verify the integrity and authenticity of the one or more data packets received by the computing device.

The techniques of this disclosure may provide one or more advantages. For example, by signing data packets that are transmitted to computing devices, an IMD may enable computing devices that receive data packets from the IMD to be able to verify the identity of the IMD that transmitted the data packets and to verify the integrity of the data packets received by the computing device, thereby ensuring the integrity and authenticity of the data received from an IMD. Ensuring the integrity and authenticity of the data received from an IMD may enable computing devices that receive such data to trust that the data received is from a particular IMD and that the data has not been changed or otherwise compromised.

In one example, a method includes generating, by processing circuitry of a medical device, one or more data packets; signing, by the processing circuitry of a medical device, one or more data packets using a private key stored in the medical device to generate a digital signature associated with the one or more data packets; and sending, by the processing circuitry, the one or more data packets and the digital signature to a computing device.

In another example, a medical device comprises: memory configured to store a private key; communication circuitry; and processing circuitry electrically coupled to the communication circuitry and the memory, wherein the processing circuitry is configured to: generate one or more data packets; sign the one or more data packets using the private key to generate a digital signature associated with the one or more data packets; and send, using the communication circuitry, the one or more data packets and the digital signature to a computing device.

In another example, an apparatus comprises: means for generating one or more data packets; means for signing the one or more data packets using a private key to generate a digital signature associated with the one or more data packets; and means for sending the one or more data packets and the digital signature to a computing device.

In another example, a non-transitory computer-readable storage medium comprising program instructions that, when executed by processing circuitry of a medical device, cause the processing circuitry to: generate one or more data packets; sign the one or more data packets using a private key to generate a digital signature associated with the one or more data packets; and send the one or more data packets and the digital signature to a computing device.

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF DRAWINGS

FIG. 1 illustrates the environment of an example medical device system in conjunction with a patient and a heart of the patient, in accordance with one or more techniques of this disclosure.

FIG. 2 is a block diagram illustrating an example configuration of components of an implantable medical device (IMD), in accordance with one or more techniques of this disclosure.

FIG. 3 is a block diagram illustrating an example configuration of components of an example computing device, in accordance with one or more techniques of this disclosure.

FIG. 4 is a flow diagram illustrating an example operation of an example implantable medical device to perform a firmware upgrade in accordance with one or more techniques of this disclosure.

FIG. 5 is a flow diagram illustrating an example operation of an example implantable medical device to perform a certificate upgrade in accordance with one or more techniques of this disclosure.

FIG. 6 is a flow diagram illustrating an example operation in accordance with one or more techniques of this disclosure.

Like reference characters denote like elements throughout the description and figures.

### DETAILED DESCRIPTION

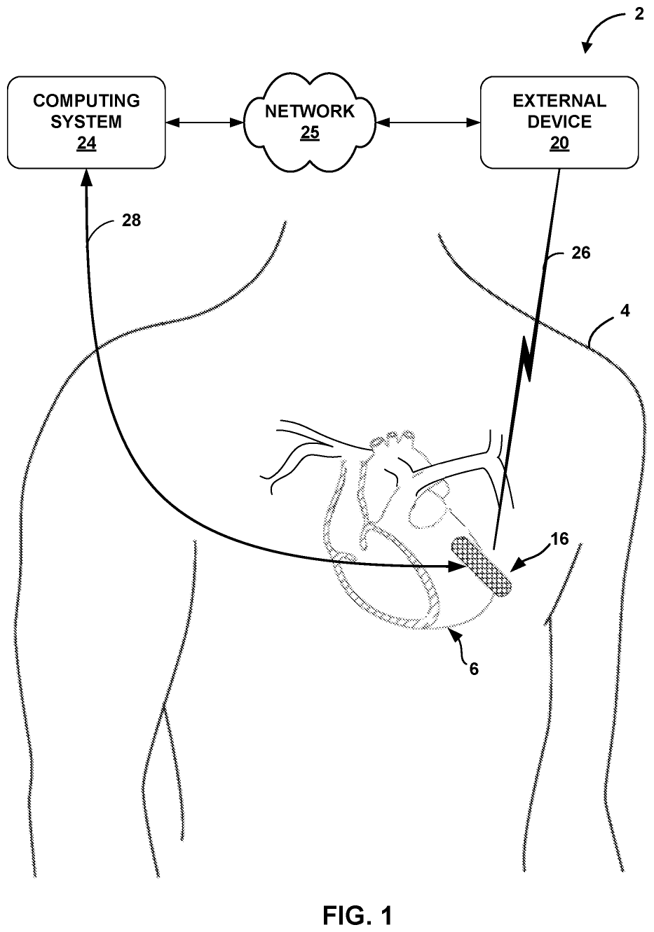

FIG. 1 illustrates the environment of an example medical device system 2 in conjunction with a patient 4 and a heart 6 of patient 4, in accordance with one or more techniques of this disclosure. The example techniques may be used with an IMD 16, which may be in wireless communication with external device 20. External device 20 may also communicate with one or more external computing system(s), such as computing system 24, via network 25.

In some examples, IMD 16 is implanted outside of a thoracic cavity of patient 4 (e.g., subcutaneously in the pectoral location illustrated in FIG. 1). IMD 16 may be positioned near the sternum near or just below the level of heart 6, e.g., at least partially within the cardiac silhouette. In the example of FIG. 1, IMD 16 is an implantable cardiac monitor, such as a LINQ™ or LINQ II™ Insertable Cardiac Monitor (ICM), available from Medtronic, Inc. of Minneapolis, Minnesota. Although in one example IMD 16 takes the form of an ICM, in other examples, IMD 16 takes the form of any combination of implantable medical device, such as an implantable cardioverter defibrillators (ICDs), pacemakers, cardiac resynchronization therapy devices (CRT-Ds), spinal cord stimulation (SCS) devices, deep brain stimulation (DBS) devices, left ventricular assist devices (LVADs), implantable sensors, orthopedic devices, or drug pumps, as examples. IMD 16 may, in some examples, be only partially implanted and/or wearable. Moreover, techniques of this disclosure may be used to communicate with any one of the aforementioned IMDs. Moreover, techniques described in this disclosure may be applied to send and receive physiological data associated with patient 4 between two or more devices, where none of the two or more devices are implantable devices. Additionally, in some examples, techniques described in this disclosure may be applied to send and receive physiological data associated with patient 4 between two or more devices, where none of the two or more devices are medical devices.

In some examples, IMD 16 includes a plurality of electrodes. The plurality of electrodes are configured to detect signals that enable processing circuitry of IMD 16 to determine current values of additional parameters associated with the cardiac and/or lung functions of patient 4. In some examples, the plurality of electrodes of IMD 16 are configured to detect a signal indicative of an electric potential of the tissue surrounding the IMD 16. Moreover, IMD 16 may additionally or alternatively include one or more accelerometers, temperature sensors, chemical sensors, light sensors, and/or pressure sensors, in some examples.

External device 20 is configured to wirelessly communicate with IMD 16 as needed to provide or retrieve information. In some examples, external device 20 acts as an external programming device, e.g., medical device programmer, for IMD 16 or as an external patient monitoring device. External device 20 is an external computing device that a user, e.g., the clinician and/or patient 4, may use to communicate with IMD 16. For example, external device 20 may communicate with IMD 16 to retrieve information from IMD 16, such as the information sensed by one or more sensors of IMD 16, settings of IMD 16, device diagnostic data (e.g., battery level, etc.). In other instances, external device 20 transmits information to IMD 16 to update one or more settings of IMD 16, such as the programmed settings that control operation of the device, updated software or firmware, or the like.

External device 20 may be a hand-held computing device (e.g., a mobile computing device) with a display viewable by the user and an interface for providing input to external device 20 (i.e., a user input mechanism). For example, external device 20 may include a small display screen (e.g., a liquid crystal display (LCD) or a light emitting diode (LED) display) that presents information to the user. In addition, external device 20 may include a touch screen display, keypad, buttons, a peripheral pointing device, voice activation, or another input mechanism that allows the user to navigate through the user interface of external device 20 and provide input. If external device 20 includes buttons and a keypad, the buttons may be dedicated to performing a certain function, e.g., a power button, the buttons and the keypad may be soft keys that change in function depending upon the section of the user interface currently viewed by the user, or any combination thereof.

In other examples, external device 20 may be a larger workstation or a separate application within another multi-function device, rather than a dedicated computing device. For example, the multi-function device may be a notebook computer, tablet computer, workstation, one or more servers, cellular phone, smart phone, personal digital assistant, or another computing device that may run an application that enables the computing device to operate as a secure device. In some examples, external device 20 may be any remote monitoring device (e.g., a smart phone, a home communicator, a monitor, etc.) that is configured to perform remote monitoring of IMD 16. In some examples, a wireless adapter coupled to the computing device enables external device 20 to establish a wireless communication link 26, such as a Bluetooth Low Energy connection, between the computing device and IMD 16. Although external device 20 is illustrated in FIG. 1 as being a single device, in some examples (not illustrated), multiple external devices may perform the functions of external device 20.

When external device 20 is configured for use by the clinician, external device 20 may be used to transmit instructions to IMD 16. Example instructions may include requests to set electrode combinations for sensing and any other information that may be useful for programming into IMD 16. The clinician may also configure and store operational parameters for IMD 16 within IMD 16 with the aid of external device 20. In some examples, external device 20 assists the clinician in the configuration of IMD 16 by providing a system for identifying potentially beneficial operational parameter values.

Whether external device 20 is configured for clinician or patient use, external device 20 is configured to communicate with IMD 16 via wireless communication, such as via communication link 26. External device 20, for example, may communicate via near-field communication technologies (e.g., inductive coupling, NFC or other communication technologies operable at ranges less than 10-20 cm) and far-field communication technologies (e.g., RF telemetry according to the 802.11, Bluetooth, or Bluetooth Low Energy specification sets, or other communication technologies operable at ranges greater than near-field communication technologies).

External device 20 may also be configured to communicate with computing system 24 via network 25. Computing system 24 may comprise computing devices configured to allow a user to interact with IMD 16, or data collected from IMD 16, via network 25. For example, computing system 24 may include one or more handheld computing devices, computer workstations, servers or other networked computing devices. In some examples, computing system 24, network 25, and external device 20 may be implemented by the Medtronic Carelink™ Network or other patient monitoring system. In some examples, external device 20 may be configured to receive data from IMD 16, e.g., daily or otherwise according to a schedule, and transmit the data to computing system 24 via network 25. In some examples, computing system 24 may be a cloud-based computing system.

Network 25 may include one or more computing devices (not shown), such as one or more non-edge switches, routers, hubs, gateways, security devices such as firewalls, intrusion detection, and/or intrusion prevention devices, servers, computer terminals, laptops, printers, databases, wireless mobile devices such as cellular phones or personal digital assistants, wireless access points, bridges, cable modems, application accelerators, or other network devices. Network 25 may include one or more networks administered by service providers, and may thus form part of a large-scale public network infrastructure, e.g., the Internet. Network 25 may provide computing devices, such as external device 20, computing system 24, and IMD 16, access to the Internet, and may provide a communication framework that allows the computing devices to communicate with one another. In some examples, network 25 may be a private network that provides a communication framework that allows computing system 24, IMD 16, and/or external device 20 to communicate with one another but isolates one or more of computing system 24, IMD 16, or external device 20 from devices external to network 25 for security purposes. In some examples, the communications between computing system 24, IMD 16, and external device 20 are encrypted.

IMD 16 may be configured to communicate with computing system 24 via external device 20 or via communication link 28. Examples of communication link 28 between IMD 16 and computing system 24 may include any suitable network connection, such as a Wi-Fi connection. In some examples, communication link 28 may include one or more networks administered by service providers, and may thus form part of a large-scale public network infrastructure, e.g., the Internet.

In general, IMD 16, external device 20, and/or computing system 24 may exchange information using at least one communication protocol. Communication protocols define sets of rules that define one or more aspects of data exchange between two or more entities of a network. In some examples, communication protocols are stored as lists of computer-readable instructions and communication protocols may be executed by any combination of hardware (e.g., physical circuitry) and software. An organization, such as a medical device manufacturer, may create its own communication protocols, license communication protocols from a third party, use open source communication protocols, or perform any combination thereof. In some examples, a communication protocol includes security provisions, such as password requirements and data encryption in order to secure the transfer of data between two or more devices in a network.

Such information exchanged between IMD 16 and external device 20 and/or information exchanged between IMD 16 and computing system 24 may include information collected/sensed by IMD 16, such as sensed physiological or biometric data from patient 4, diagnostic determinations made based on the sensed physiological or biometric data, therapy data associated with a therapy delivered to patient 4, performance data regarding operation and performance of IMD 16 (e.g., power level information, information regarding strengths of signals received, information regarding frequency of received interrogation requests, remaining battery life, etc.). The information may also include information sent by external device 20 to IMD 16, such as instructions, such as requests to set electrode combinations for sensing, and/or any other information (e.g., operational parameter values) that may be useful for programming into IMD 16.

IMD 16 and external device 20 may establish a wireless connection between IMD 16 and external device 20, such as in the form of communication link 26, in order to exchange information using at least one communication protocol. To establish a wireless connection between IMD 16 and external device 20, IMD 16 and external device 20 may perform a pairing process, during which IMD 16 and external device 20 may exchange information to form a trusted relationship prior to being able to communicate certain information with one another. In the example where IMD 16 and external device 20 attempt to establish a Bluetooth connection, IMD 16 and external device 20 may perform a pairing process according to Bluetooth specifications. Similarly, in the example where IMD 16 and external device 20 attempt to establish a Bluetooth Low Energy connection, IMD 16 and external device 20 may perform a pairing process according to Bluetooth Low Energy specifications.

IMD 16 may send information or other data to external device 20 and/or computing system 24 in the form of data packets. The data packets sent by IMD 16 may be of any suitable form and contain any suitable data structure that includes any suitable information, such as the examples of information described throughout this disclosure.

In accordance with aspects of this disclosure, IMD 16 may include (i.e., store in the memory of IMD 16), a private key. IMD 16 may generate data packets, which may include any suitable information, such as physiological data of patient 4 sensed by IMD 16, and IMD 16 may use the private key to digitally sign data packets to be transmitted to external device 20 and/or computing system 24. That is, IMD 16 may, for each data packet or for a group of data packets, generate an associated digital signature using the private key based on the data packet or the group of data packets. For example, IMD 16 may digitally sign a data packet by generating a digest of the data packet, such as by performing a one-way hash of the data packet, and by using the private key to encrypt the digest of the data packet to generate the digital signature associated with the data packet.

IMD 16 may therefore transmit data packets and the associated digital signature of each of the data packets to external device 20 and/or computing system 24. External device 20 and/or computing system 24 may, in response to receiving the data packets and the associated digital signatures, verify the integrity of the data packets and/or verify the identity of the sender of the data packets using the associated digital signatures. External device 20 and/or computing system 24 may generate a digest of a data packet received from IMD 16 and may correspondingly decrypt the digital signature received from IMD 16, and may compare the digest with the decrypted digital signature. If external device 20 and/or computing system 24 determines that the digest and the decrypted digital signature match (e.g., have the same value), external device 20 and/or computing system 24 may determine that the integrity of the data packet and the identity of the sender of the data packet have successfully been verified.

In some examples, IMD 16 may include (i.e., store in the memory of IMD 16) a digital certificate associated with IMD 16, such as a digital certificate having a format that conforms to the X.509 standard (also referred to as an X.509 certificate). The digital certificate is also referred to as a public key certificate. The digital certificate may bind an identity, such as the identity of IMD 16, and a public key, and the digital certificate may be signed by a certificate authority or may be self-signed. If the digital certificate is self-signed, such as by using a private key of IMD 16, the digital certificate may be a root certificate. For example, IMD 16 may include a root certificate that is self-signed using a private key used by IMD 16 to sign data packets that are transmitted by IMD 16.

The digital certificate associated with IMD 16 may be distributed to external device 20 and/or computing system 24 that communicate with IMD 16, so that each of external device 20 and/or computing system 24 may store a copy of the digital certificate associated with IMD 16. Because the digital certificate associated with IMD 16 includes the public key that corresponds to IMD 16's private key that is used by IMD 16 to sign data packets transmitted to external device 20 and/or computing system 24, external device 20 and/or computing system 24 may, by receiving the digital certificate associated with IMD 16, receive a public key that can be used to verify the authenticity of the data packets received from IMD 16.

In some examples, prior to sending data packets that are signed using IMD 16's private key, IMD 16 may distribute (e.g., transmit) a digital certificate associated with IMD 16 to external device 20 and/or computing system 24. External device 20 and/or computing system 24 may use the digital certificate received from IMD 16 to verify that the public key included in the digital certificate received from IMD 16 was issued by IMD 16. External device 20 and/or computing system 24 may, in response to successfully verifying that the public key included in the digital certificate received from IMD 16 was issued by IMD 16, store the public key in memory for use in verifying the integrity of the data packets received from IMD 16 and for use in verifying the identity of the sender of the data packets.

In some examples, IMD 16 may perform a firmware upgrade on the firmware of IMD 16's communication circuitry, such as performing a firmware upgrade on the firmware of IMD 16's Bluetooth radio. To perform such a firmware upgrade, IMD 16 may receive (e.g., from external device 20) firmware upgrade data, such as in the form of a firmware upgrade image, and may store the firmware upgrade data in a sparse location in the memory of IMD 16. IMD 16 may disable IMD 16's communication circuitry that is to be upgraded, such as by disabling IMD 16's Bluetooth radio. IMD 16 may verify the integrity of the firmware of IMD 16's communication circuitry, such as verifying the integrity of the firmware of IMD 16's Bluetooth radio. IMD 16 may also verify the integrity of the firmware upgrade data. If IMD 16 is able to successfully verify both the integrity of the firmware of IMD 16's communication circuitry as well as the integrity of the firmware upgrade data, IMD 16 may apply the firmware upgrade data to the firmware of IMD 16's communication circuitry to upgrade the firmware of IMD 16's communication circuitry.

IMD 16 may verify the integrity of the firmware upgrade data by performing code signature verification of the firmware upgrade data. By performing code signature verification of the firmware upgrade data, IMD 16 may be able to verify that the firmware upgrade data is from a trusted source, that the firmware upgrade data is applicable to the specific IMD 16, and/or that the firmware upgrade data is intended for patient 4.

In some examples, IMD 16 may receive a firmware upgrade package that includes the firmware upgrade data and a code signature associated with the firmware upgrade data, and IMD 16 may verify the integrity of the firmware upgrade data based at least in part on verifying the code signature associated with the firmware upgrade data. Such a code signature may be a result of external device 20 and/or computing system 24 digitally signing the firmware upgrade data using a private key to generate the code signature. IMD 16 may therefore use a public key that corresponds to the private key to verify the code signature in order to verify the integrity of the firmware upgrade data.

Similarly, in some examples, IMD 16 may also perform a certificate upgrade to upgrade the digital certificates stored in IMD 16. Performing a certificate upgrade may include replacing the digital certificate stored in IMD 16 with an upgraded digital certificate or may include modifying the digital certificate stored in IMD 16 to generate an upgraded digital certificate in IMD 16. IMD 16 may receive certificate upgrade data, which may include data for upgrading a digital certificate stored in IMD 16, from external device 20 and/or computing system 24. The certificate upgrade data may include an upgraded digital certificate or may include code for modifying the digital certificate stored in IMD 16 to generate an upgraded digital certificate in IMD 16.

IMD 16 may verify the integrity of the certificate upgrade data based at least in part on verifying a digital signature associated with the certificate upgrade data. Such a digital signature may be a result of external device 20 and/or computing system 24 digitally signing the certificate upgrade data using a private key to generate the digital signature. IMD 16 may therefore use a public key that corresponds to the private key to verify the digital signature in order to verify the integrity of the certificate upgrade data.

In some examples, in addition to the digital signature or in the alternative, the certificate upgrade package received by IMD 16 may also include a digital certificate or other form of data that indicates the source of the certificate upgrade data, the intended IMD to be upgraded by the certificate upgrade data, and/or the patient having the IMD that is to be upgraded by the certificate upgrade data. IMD 16 may verify the integrity of the certificate upgrade data based at least in part on verifying the digital certificate associated with the certificate upgrade data. The digital certificate may include a digital signature signed using a private key. IMD 16 may therefore use a public key that corresponds to the private key to verify the digital signature in order to verify the source of the certificate upgrade data, the intended IMD to be upgraded by the certificate upgrade data, and/or the patient having the IMD that is to be upgraded by the certificate upgrade data specified by the digital certificate.

In some examples, if every root certificate stored in IMD 16 is expired, IMD 16 may relax signature verification requirements in order to update a digital certificate stored in IMD 16 as long as the digital certificate is previously valid prior to its expiration. Thus, if all root certificates in IMD 16 are expired, external device 20 and/or computing system 24 may digitally sign the certificate upgrade data using a private key associated with an expired root certificate in IMD 16, and IMD 16 may be able to verify the digital signature of the certificate upgrade data using a corresponding public key associated with the expired root certificate.

In some examples, if every root certificate stored in IMD 16 is expired, external device 20 and/or computing system 24 may send a certificate upgrade package signed using an expired root certificate via an out-of-band communication link. If IMD 16 and external device 20 are wirelessly connected and communicate via communication link 26, external device 20 may, in response to determining that every root certificate stored in IMD 16 is expired, send a certificate upgrade package signed using an expired root certificate via a communication link different from communication link 26. Such a communication link may implement a different communication protocol than communication link 26. For example, if communication link 26 is a Bluetooth communication link, external device 20 may transmit, to IMD 16, the certificate upgrade package via a Wi-Fi communication link or another non-Bluetooth communication link.

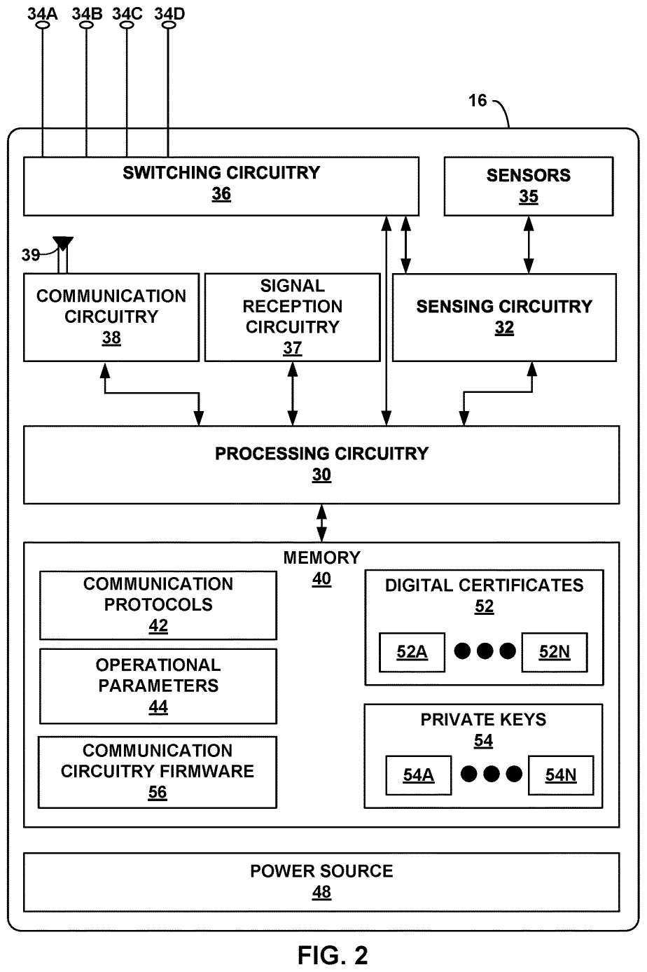

FIG. 2 is a block diagram illustrating an example configuration of components of IMD 16 in accordance with one or more techniques of this disclosure. In the example of FIG. 2, IMD 16 includes processing circuitry 30, sensing circuitry 32, electrodes 34A-34D (collectively, “electrodes 34”), sensors 35, switching circuitry 36, signal reception circuitry 37, communication circuitry 38, antenna 39, memory 40, and power source 48. Memory 40 is configured to store communication protocols 42, operational parameters 44, and/or collected physiological data.

Power source 48 is configured to deliver operating power to the components of IMD 16. Power source 48 may include a battery and a power generation circuit to produce the operating power. In some examples, the battery is rechargeable to allow extended operation. In some examples, recharging is accomplished through proximal inductive interaction between an external charger and an inductive charging coil within external device 18. Power source 48 may include any one or more of a plurality of different battery types, such as nickel cadmium batteries and lithium ion batteries.

Processing circuitry 30, in one example, may include one or more processors that are configured to implement functionality and/or process instructions for execution within IMD 16. For example, processing circuitry 30 may be capable of processing instructions stored in memory 40. Processing circuitry 30 may include, for example, microprocessors, digital signal processors (DSPs), application specific integrated circuits (ASICs), field-programmable gate arrays (FPGAs), or equivalent discrete or integrated logic circuitry, or a combination of any of the foregoing devices or circuitry. Accordingly, processing circuitry 30 may include any suitable structure, whether in hardware, software, firmware, or any combination thereof, to perform the functions ascribed herein to processing circuitry 30.

Sensing circuitry 32 monitors electrical cardiac signals from any combination of electrodes 34A-34D (collectively, “electrodes 34”). In some examples, sensing circuitry 32 may include one or more amplifiers, filters, and analog-to-digital converters. For example, sensing circuitry 32 may include one or more detection channels, each of which may include an amplifier. The detection channels may be used to sense cardiac signals, such as a cardiac EGM. Some detection channels may detect events, such as R-waves, P-waves, and T-waves and provide indications of the occurrences of such events to processing circuitry 30. Additionally, or alternatively, some channels may detect cardiac EGM signals from a particular combination of electrodes 34. One or more other detection channels may provide signals to an analog-to-digital converter, for conversion into a digital signal for processing, analysis, storage, or output by processing circuitry 30.

Each detection channel of sensing circuitry 32 may include a filter configured to pass a custom range of frequency values. For example, sensing circuitry 32 may include one or more narrow band channels, each of which may include a narrow band filtered sense-amplifier. Additionally, or alternatively, sensing circuitry 32 may include one or more wide band channels, each of which include an amplifier with a relatively wider pass band than the narrow band channels. Signals sensed by the narrow band channels and the wide band channels of sensing circuitry 32 may be converted to multi-bit digital signals by an analog-to-digital converter (ADC) provided by, for example, sensing circuitry 32 or processing circuitry 30. In some examples, processing circuitry 30 analyzes the digitized version of signals from sensing circuitry 32.

Processing circuitry 30 may use switching circuitry 36 to select, e.g., via a data/address bus, which of electrodes 34 to use for sensing cardiac signals. Switching circuitry 36 may include a switch array, switch matrix, multiplexer, or any other type of switching device suitable to selectively couple energy to selected electrodes.

In some examples, sensing circuitry 32 is electrically coupled to sensors 35. Sensors 35 may include any combination of accelerometers, temperature sensors, chemical sensors, light sensors, and pressure sensors. Sensors 35 may, for example, sense one or more physiological parameters indicative of a heart condition. Additionally, or alternatively, an accelerometer of sensors 35 may sense data indicative of at least one of patient posture and patient activity.

Signal reception circuitry 37 may include hardware, firmware, software or any combination thereof for receiving signals from another device, such as external device 20. Signal reception circuitry 37 may be coupled to communication circuitry 38 and/or antenna 39, and may be powered by power source 48 (e.g., periodically), “listening” for signals from external device 20. In this way, signal reception circuitry 37 may alternate between an “off” state and an “on” state, where signal reception circuitry 37 is configured to detect signals while signal reception circuitry 37 is being powered by power source 48 during the on state.

Communication circuitry 38 may include any suitable hardware, firmware, software or any combination thereof for communicating with another device, such as external device 20 and/or computing system 24. Under the control of processing circuitry 30, communication circuitry 38 may receive downlink telemetry from, as well as send uplink telemetry to, external device 20 or another device with the aid of an internal or external antenna, e.g., antenna 39, and/or signal reception circuitry, e.g., signal reception circuitry 37. In addition, processing circuitry 30 may communicate with a networked computing device via an external device (e.g., external device 20) and a computer network, such as the Medtronic CareLink® Network developed by Medtronic, plc, of Dublin, Ireland. Communication circuitry 38 may include any combination of a Bluetooth radio, a Wi-Fi radio, an electronic oscillator, frequency modulation circuitry, frequency demodulation circuitry, amplifier circuitry, and power switches such as a metal-oxide-semiconductor field-effect transistors (MOSFET), a bipolar junction transistor (BJT), an insulated-gate bipolar transistor (IGBT), a junction field effect transistor (JFET), or another element that uses voltage for its control. Signal reception circuitry 37 may, in some cases, be separate from communication circuitry 38. In other cases, signal reception circuitry 37 may be a component of, or a part of communication circuitry 38.

Memory 40 may be configured to store information within IMD 16 during operation. Memory 40 may include a computer-readable storage medium or computer-readable storage device. In some examples, memory 40 includes one or more of a short-term memory or a long-term memory. Memory 40 may include, for example, random access memories (RAM), dynamic random access memories (DRAM), static random access memories (SRAM), magnetic discs, optical discs, flash memories, or forms of electrically programmable memories (EPROM) or electrically erasable and programmable memories (EEPROM). In some examples, memory 40 is used to store data indicative of instructions for execution by processing circuitry 30.

In some examples, memory 40 is configured to store one or more communication protocols 42. Each protocol of communication protocols 42 may define a set of rules that govern one or more aspects of data exchange between IMD 16 and other devices (e.g., external device 20 and computing system 24). In some examples, communication protocols 42 are stored as lists of computer-readable instructions and communication protocols may be executed by any combination of hardware (e.g., processing circuitry 30) and software. In some examples, communication protocols 42 include a Bluetooth® protocol such as a BTLE protocol. In some examples, communication protocols 42 exclusively include the Bluetooth® protocol. Alternatively, in other examples, communication protocols 42 may include any combination of Bluetooth® protocols, protocols developed by the manufacturer of IMD 16, and protocols licensed from a third-party developer.

In some examples, memory 40 is configured to store operational parameters 44. Operational parameters 44 may govern aspects of the operation of IMD 16. For example, operational parameters 44 may include combinations of electrodes 34 and sensors 35 for sensing physiological signals of patient 4. Additionally, or alternatively, operational parameters 44 may include a sampling rate for sampling analog signals sensed by electrodes 34 and sensors 35. Operational parameters 44 may be updated based on instructions received from an external device (e.g., external device 20) via communication circuitry 38. In some examples, processing circuitry 30 of IMD 16 updates operational parameters 44 only if instructions to update operational parameters 44 are received over a secure link.

In some examples, memory 40 is configured to store communication circuitry firmware 56. Communication circuitry firmware 56 may be firmware for communication circuitry 38 stored in memory 40 and may be updated and/or reprogrammed. Communication circuitry firmware 56 may, for example, be stored in EPROM or EEPROM of memory 40. If communication circuitry 38 includes a Bluetooth radio, communication circuitry firmware 56 may be firmware for the Bluetooth radio.

In some examples, memory 40 is configured to store digital certificates 52A-52N (“digital certificates 52”). Each of digital certificates may have a format that conforms to the X.509 standard (also referred to as an X.509 certificate) or other public key infrastructure (PKI) standard or certificate standard. A digital certificate is also referred to as a public key certificate. Each of digital certificates may bind an identity, such as the identity of IMD 16, and a public key, so that each digital certificate may be associated with a public key.

Each of digital certificates 52 may be signed by a certificate authority or may be self-signed. As such, each digital certificate includes or is associated with a digital signature that is produced by signing the digital certificate using either a private key of a Certificate Authority or a private key of IMD 16. If a digital certificate is self-signed, such as by using a private key of IMD 16, the digital certificate may be a root certificate. Digital certificates 52 may include one or more root certificates.

In some examples, memory 40 is configured to store private keys 54A-54N (“private keys 54”) associated with digital certificates 52. Each of private keys 54 may be part of a asymmetric key pair with a corresponding public key of a corresponding digital certificate of digital certificates 52. For example, each asymmetric key pair of a private key and a corresponding public key may be an RSA key pair.

IMD 16 may establish a communication link with another device, such as external device 20 and/or computing system 24. For example, IMD 16 may receive data from external device 20 via communication circuitry 38, and IMD 16 may send data to external device 20 via communication circuitry 38. Similarly, IMD 16 may receive data from computing system 24 via communication circuitry 38, and IMD 16 may send data to computing system 24 via communication circuitry 38. IMD 16 may send and receive data according to one or more of communication protocols 42. Communication protocols 42 may include one or more protocols and may enable IMD 16 to communicate according to a Bluetooth protocol or a Bluetooth Low Energy protocol, as an example. In some examples, IMD 16 and external device 20 may exchange one or more keys (e.g., one or more public/private key pairs) as part of establishing the communication link.

Processing circuitry 30 of IMD 16 is configured to collect and/or sense (e.g., via sensing circuitry 32) data. Such data may include sensed physiological or biometric data from patient 4, diagnostic determinations made based on the sensed physiological or biometric data, therapy data associated with a therapy delivered to patient 4, performance data regarding operation and performance of IMD 16 (e.g., power level information, information regarding strengths of signals received, information regarding frequency of received interrogation requests, remaining battery life, etc.).

Processing circuitry 30 may be configured to transmit the collected and/or sensed data to external device 20 and/or computing system 24 in any suitable form, such as in the form of data packets. A data packet may be a formatted unit of data, such as a datagram, a segment, a block, a cell, a frame, and the like containing data collected and/or sensed by processing circuitry 30 and having any suitable format, structure, and/or size.

Processing circuitry 30 is configured to send the data collected and/or sensed by IMD 16 to external device 20 and/or computing system 24. Operational parameters 44 may include information for controlling the data that IMD 16 sends to external device 20 and/or computing system 24 and for controlling the recipient of the collected/sensed data. For example, processing circuitry 30 may determine, based on operational parameters 44, which of the data collected/sensed by IMD 16 are to be transmitted by IMD 16 and the destination (e.g., external device 20 and/or computing system 24) to which IMD 16 may transmit the data.

Processing circuitry 30 is configured to digitally sign, using a private key of private keys 54, one or more data packets that are sent, via communication circuitry 38, to external device 20 and/or computing system 24. Processing circuitry 30 may be configured to sign one or more data packets by using a private key of private keys 54 to generate a digital signature associated with the one or more data packets. Processing circuitry 30 may be configured to use a private key of private keys 54 to sign one or more data packets using any suitable digital signature scheme, such as an RSA-based digital signature scheme, a Lamport one-time signature scheme, a Merkle signature scheme, a Rabin signature algorithm, and the like. For example, to sign one or more data packets, processing circuitry 30 may be configured to hash one or more data packets to create a digest, to pad the digest, and to sign the padded digest using a private key to generate a digital signature for the one or more data packets.

In some examples, processing circuitry 30 may digitally sign each individual data packet transmitted by IMD 16. That is, processing circuitry 30 may generate a digital signature for each data packet. In some examples, processing circuitry 30 may digitally sign a group of data packets. That is, processing circuitry 30 may generate a single digital signature for a group of data packets. In examples where processing circuitry 30 may digitally sign a group of data packets, processing circuitry 30 may be configured to group two or more data packets into a group in any suitable manner. In some examples, processing circuitry 30 may be configured to group consecutive data packets to be sent to the same destination (e.g., consecutive data packets to be sent to external device 20) in a group and may generate a single digital signature for the group of data packets. In some examples, processing circuitry 30 may be configured to group consecutive data packets to be sent to the same destination if each data packet contains the same type of data. For example, processing circuitry 30 may be configured to group consecutive data packets that each include physiological data, but may be configured to refrain from grouping consecutive data packets that includes a mix of physiological data and operational data regarding IMD 16.

As described above, IMD 16 may include a plurality of private keys 54 that correspond to a plurality of digital certificates 52. Processing circuitry 30 may therefore be configured to select a private key from the plurality of private keys 54 to encrypt one or more data packets. Each destination for data packets transmitted by IMD 16 may store one of IMD's digital certificates 52, so that the destination (e.g., external device 20 or computing system 24) may use the public key associated with the digital certificate to verify the digital signatures associated with packets sent from IMD 16 to the destination.

Different destinations for data packets transmitted by IMD 16 may have the same or different digital certificates of digital certificates 52. For example, external device 20 may have a digital certificate and an associated public key that is different from the digital certificate and associated public key stored at computing system 24. As such, processing circuitry 30 may be configured to select a private key to encrypt one or more data packets from private keys 54 based at least in part on the destination of the one or more data packets. Specifically, memory 40 may store associations between destinations (i.e., remote computing devices and/or systems) and digital certificates 52, such that the associations indicate, for each destination, the digital certificate stored at the destination. Processing circuitry 30 may therefore be configured to determine, based on the associations between destinations and digital certificates, the private key from private keys 54 to use for digitally signing one or more data packets to be sent to a particular destination.

In some examples, different types of destinations may store different digital certificates associated with different public keys. As such, processing circuitry 30 may be configured to select the private key from private keys 54 for digitally signing one or more data packets based on the type of destination for the one or more data packets. For example, a patient monitor, such as external device 20, that communicates with IMD 16 via short-range wireless communications (e.g., Bluetooth) may store a digital certificate from digital certificates 52 that is different from the digital certificate stored by a computing system (e.g., computing system 24) that communicates with IMD 16 via a LAN (e.g., over the Internet) or that communicates with IMD 16 using external device 20 as an intermediary.

In another example, different destinations in different geographic regions may store different digital certificates associated with different public keys. For example, computing devices in different countries may store different digital certificates associated with different public keys, such as public keys that use different encryption algorithms and/or public keys having different encryption strengths. As such, processing circuitry 30 may be configured to select the private key from private keys 54 for digitally signing one or more data packets based on the geographic location of the destination for the one or more data packets.

In some examples, processing circuitry 30 may be configured to receive, via communication circuitry 38, firmware upgrade data for performing a firmware upgrade on communication circuitry firmware 56, which is firmware for communication circuitry 38. For example, IMD 16 may be configured to perform a firmware upgrade on communication circuitry firmware 56 of a Bluetooth radio in communication circuitry 38. Processing circuitry 30 may be configured to receive, via communication circuitry 38 and from an external device (e.g., external device 20 or computing system 24), firmware upgrade data, such as in the form of a firmware upgrade image, and may store the firmware upgrade data in a sparse location in memory 40.

In some examples, processing circuitry 30 may be configured to be able to receive, via communication circuitry 38, the firmware upgrade data from an external device (e.g., external device 20 or computing system 24) over multiple different connections with the external device over time (e.g., over a number of days or weeks). Processing circuitry 30 may be configured to establish a communication link (e.g., communication link 26 or communication link 28) between IMD 16 and the external device to download the firmware upgrade data. If the communication link is disconnected before processing circuitry 30 completes download of the firmware upgrade data, processing circuitry 30 may be able to reestablish a communication link between IMD 16 and the external device, such as when the external device is back within range of IMD 16, to continue download of the firmware upgrade data. In this way, if the communication link between IMD 16 and the external device continues to be disconnected before processing circuitry 30 finishes downloading the firmware upgrade data, processing circuitry 30 may be able to periodically reestablish a communication link with the external device, over hours, days, weeks, and the like, to continue downloading the firmware upgrade data until processing circuitry 30 is able to finish downloading the firmware upgrade data.

In some examples, processing circuitry 30 may be configured to be able to receive, via communication circuitry 38, the firmware upgrade data from multiple different external device (e.g., from external device 20 and computing system 24) over multiple different connections with the multiple external device over time (e.g., over a number of days or weeks). For example, processing circuitry 30 may be configured to establish a communication link 26 between IMD 16 and external device 20 to download the firmware upgrade data. If communication link 26 is disconnected before processing circuitry 30 completes download of the firmware upgrade data, processing circuitry 30 may be able to reestablish a communication link with external device 20 or with computing system 24 to continue download of the firmware upgrade data. For example, after communication link 26 between IMD 16 and external device 20 is disconnected, processing circuitry 30 may determine that IMD 16 is able to establish a communication link with computing system 24 to resume downloading of the firmware upgrade data. Processing circuitry 30 may therefore be configured to establish communication link 28 between IMD 16 and computing system 24 to resume downloading of the firmware upgrade data.

Processing circuitry 30 may be configured to verify the integrity and authenticity of communication circuitry firmware 56 as well as the integrity of the firmware upgrade data. Processing circuitry 30 may be configured to verify the integrity of communication circuitry firmware 56 via any suitable technique, such as by generating a hash of communication circuitry firmware 56 and comparing the hash of communication circuitry firmware 56 against a previously-determined hash of communication circuitry firmware 56.

Processing circuitry 30 may be configured to verify the integrity of the firmware upgrade data by performing code signature verification of the firmware upgrade data. By performing code signature verification of the firmware upgrade data, processing circuitry 30 may be able to verify the integrity of the firmware upgrade data. In some examples, processing circuitry 30 may be configured to receive, via communication circuitry 38, a firmware upgrade package that includes the firmware upgrade data and a code signature associated with the firmware upgrade data, and processing circuitry 30 may be configured to verify the integrity of the firmware upgrade data based at least in part on verifying the code signature associated with the firmware upgrade data. Such a code signature may be a result of external device 20 and/or computing system 24 digitally signing the firmware upgrade data using a private key that corresponds to a digital certificate out of digital certificates 52 stored in memory 40 to generate the code signature. Processing circuitry 30 may therefore be configured to use a public key that corresponds to the digital certificate out of digital certificates 52 to verify the code signature in order to verify the integrity of the firmware upgrade data.

In some examples, the firmware upgrade package may include a digital certificate associated with the firmware upgrade data that may specify information such as the source of the firmware upgrade data, the IMD(s) to which the firmware upgrade data is applicable, and/or the patient(s) for which the firmware upgrade data is intended. By verifying the digital certificate, processing circuitry 30 may be able to verify that the firmware upgrade data is from a trusted source, that the firmware upgrade data is applicable to the specific IMD 16, and/or that the firmware upgrade data is intended for patient 4.

In some examples, the digital certificate included in the firmware upgrade package is signed using a private key that corresponds to a digital certificate out of digital certificates 52 stored in memory 40 to generate the certificate signature. That is, the digital certificate may include or be associated with a digital signature that is a result of signing the digital certificate using such a private key. Processing circuitry 30 may therefore be configured to use a public key that corresponds to the digital certificate out of digital certificates 52, which corresponds to the private key used to sign the digital certificate to verify the digital certificate in order to verify whether the firmware upgrade data is from a trusted source, whether the firmware upgrade data is applicable to the specific IMD 16, and/or whether the firmware upgrade data is intended for patient 4.

Processing circuitry 30 may be configured to, in response to successfully verifying the code signature associated with the firmware upgrade data and/or successfully verifying the digital certificate associated with the firmware upgrade data, upgrade communication circuitry firmware 56 using the firmware upgrade data. Processing circuitry 30 may be configured to disable communication circuitry 38 that is to be upgraded and to apply the firmware upgrade data to communication circuitry firmware 56 to upgrade communication circuitry firmware 56.

In some examples, processing circuitry 30 may also be configured to perform a certificate upgrade to upgrade one or more of digital certificates 52 stored in IMD 16. Performing a certificate upgrade may include replacing a digital certificate 52 stored in IMD 16 with an upgraded digital certificate 52 or may include modifying a digital certificate 52 stored in IMD 16 to generate an upgraded digital certificate in IMD 16. Upgrading a digital certificate 52 may include replacing an expired digital certificate with an unexpired digital certificate or upgrading an expired digital certificate so that the digital certificate, after being upgraded, is no longer expired.

Processing circuitry 30 may be configured to receive certificate upgrade data, which may include data for upgrading a digital certificate stored in memory 40, such as digital certificate 52A, from external device 20 and/or computing system 24. The certificate upgrade data may include an upgraded digital certificate to replace digital certificate 52A or may include code for modifying digital certificate 52A to generate an upgraded digital certificate in memory 40.

Processing circuitry 30 may be configured to verify the integrity of the certificate upgrade data based at least in part on verifying a digital signature associated with the certificate upgrade data. Such a digital signature may be a result of external device 20 and/or computing system 24 digitally signing the certificate upgrade data using a private key which corresponds to a public key that corresponds to a digital certificate of digital certificates 52 to generate the digital signature. Processing circuitry 30 may therefore be configured to use a public key that corresponds to a digital certificate of digital certificates 52, which corresponds to the private key used to sign the certificate upgrade data to verify the digital signature in order to verify the integrity of the certificate upgrade data.

In some examples, in addition to the digital signature or in the alternative, the certificate upgrade package received by IMD 16 may also include a digital certificate or other form of data that indicates the source of the certificate upgrade data, the intended IMD to be upgraded by the certificate upgrade data, and/or the patient having the IMD that is to be upgraded by the certificate upgrade data. Processing circuitry 30 may be configured to verify the integrity of the certificate upgrade data based at least in part on verifying the digital certificate associated with the certificate upgrade data. The digital certificate may include a digital signature signed using a private key which corresponds to a public key that corresponds to a digital certificate of digital certificates 52. Processing circuitry 30 may therefore be configured to use a public key that corresponds to a digital certificate of digital certificates 52, which corresponds to the private key used to sign the digital certificate to verify the digital signature. If processing circuitry 30 is able to successfully verify the digital signature, processing circuitry 30 may therefore be able to verify the information indicated by the digital certificate, such as the source of the certificate upgrade data, the intended IMD to be upgraded by the certificate upgrade data, and/or the patient having the IMD that is to be upgraded by the certificate upgrade data specified by the digital certificate. If processing circuitry 30 is able to verify the integrity of the certificate upgrade data, processing circuitry 30 may apply the certificate upgrade data to upgrade a digital certificate stored in memory 40, such as digital certificate 52A.

In some examples, if every root certificate of digital certificates 52 stored in memory 40 is expired, processing circuitry 30 may be configured to relax signature verification requirements in order to update a digital certificate of digital certificates 52 as long as the digital certificate was previously valid prior to its expiration. Thus, if all root certificates in memory 40 are expired, external device 20 and/or computing system 24 may digitally sign the certificate upgrade data using a private key associated with an expired root certificate in memory 40, and processing circuitry 30 may be configured to verify the digital signature of the certificate upgrade data using a corresponding public key associated with the expired root certificate.

In some examples, if every root certificate stored in IMD 16 is expired, external device 20 and/or computing system 24 may send certificate upgrade package signed using an expired root certificate via an out-of-band communication link. If IMD 16 and external device 20 are wirelessly connected and communicate via communication link 26, external device 20 may, in response to determining that every root certificate stored in IMD 16 is expired, send certificate upgrade package signed using an expired root certificate via a communication link different from communication link 26. Such a communication link may implement a different communication protocol than communication link 26. For example, if communication link 26 is a Bluetooth® communication link, external device 20 may transmit, to IMD 16, the certificate upgrade package via a Wi-Fi communication link or another non-Bluetooth® communication link. In this way, IMD 16 may be able to receive a certificate upgrade package out-of-band in order to upgrade one or more expired root certificates in memory 40.

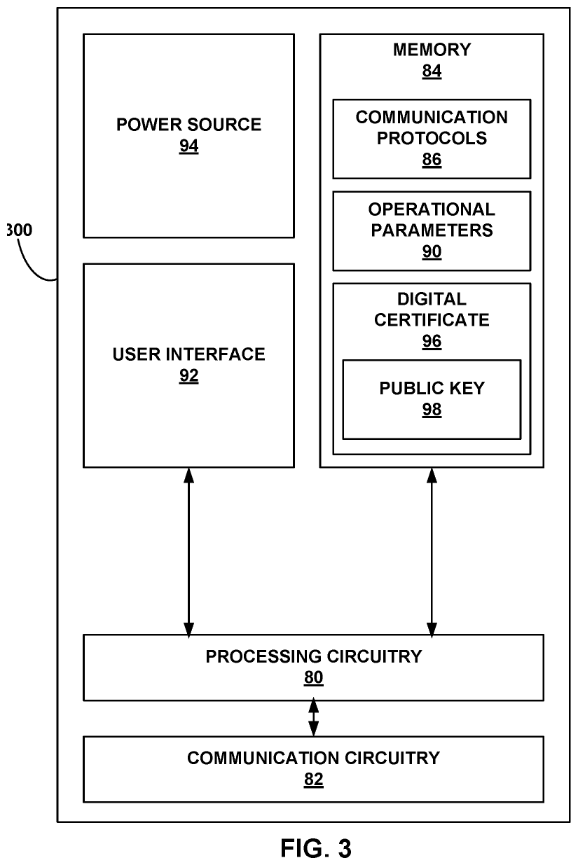

FIG. 3 is a block diagram illustrating an example configuration of components of computing device 300 in accordance with one or more techniques of this disclosure. Computing device 300 shown in FIG. 3 may be an example of external device 20 or computing system 24 of FIG. 1.

In the example of FIG. 3, computing device 300 includes processing circuitry 80, communication circuitry 82, memory 84, user interface 92, and power source 94. Memory 84 is configured to store communication protocols 86, operational parameters 90, and digital certificate 96 that includes or is associated with public key 98.

Processing circuitry 80, in one example, may include one or more processors that are configured to implement functionality and/or process instructions for execution within computing device 300. For example, processing circuitry 80 may be capable of processing instructions stored in memory 84. Processing circuitry 80 may include, for example, microprocessors, DSPs, ASICs, FPGAs, or equivalent discrete or integrated logic circuitry, or a combination of any of the foregoing devices or circuitry. Accordingly, processing circuitry 80 may include any suitable structure, whether in hardware, software, firmware, or any combination thereof, to perform the functions ascribed herein to processing circuitry 80.

Communication circuitry 82 may include any suitable hardware, firmware, software or any combination thereof for communicating with another device, such as IMD. Under the control of processing circuitry 80, communication circuitry 82 may receive uplink telemetry from, as well as send downlink telemetry to, IMD 16 or another device. In some examples, communication circuitry 82 includes a first set of communication circuitry configured for transmitting and receiving signals according to a communication protocol developed by the manufacturer of IMD 16 or a third-party developer. In some such examples, communication circuitry 82 further includes a second set of communication circuitry which defines a Bluetooth radio configured for transmitting and receiving signals according to Bluetooth communication protocols, including Bluetooth Low Energy protocols. However, communication circuitry 82 does not necessarily include separate sets of circuitry corresponding to different communication protocols. In some examples, communication circuitry 82 includes a single set of circuitry configured for transmitting and receiving signals according to a plurality of communication protocols.

In some examples, communication circuitry 82 includes any combination of a Bluetooth radio, a Wi-Fi radio, an Ethernet port, an electronic oscillator, frequency modulation circuitry, frequency demodulation circuitry, amplifier circuitry, and power switches such as a MOSFET, a BJT, an IGBT, a JFET, or another element that uses voltage for its control.

Memory 84 may be configured to store information within computing device 300 during operation. Memory 84 may include a computer-readable storage medium or computer-readable storage device. In some examples, memory 84 includes one or more of a short-term memory or a long-term memory. Memory 84 may include, for example, RAM, DRAM, SRAM, magnetic discs, optical discs, flash memories, or forms of EPROM or EEPROM. In some examples, memory 84 is used to store data indicative of instructions for execution by processing circuitry 80. Memory 84 may be used by software or applications running on computing device 300 to temporarily store information during program execution.

Computing device 300 may exchange information with other devices via communication circuitry 82 according to one or more communication protocols 86. Communication protocols 86, stored in memory 84, may include sets of computer-readable instructions that determine how data is transmitted and processed. Communication protocols 86 may include one or more communication protocols that are additionally included in communication protocols 42. In other words, IMD 16, and computing device 300 may be configured to exchange information according to at least one common communication protocol. In some examples, the one or more common communication protocols include a Bluetooth communication protocol, a Wi-Fi communication protocol, an Internet protocol, and the like. Additionally, or alternatively, communication protocols 86 may include a set of communication protocols that are not available to IMD 16. In some examples, computing device 300 is a consumer electronics device, such as a smartphone, a tablet, or a laptop computer, a desktop computer, a server computer, or a computing device that is a part of a cloud system.

Data exchanged between computing device 300 and IMD 16 may also include any of operational parameters 90 stored in memory 84. Computing device 300 may transmit data including computer readable instructions which, when implemented by IMD 16, may control IMD 16 to change one or more operational parameters according to operational parameters 90 and/or export collected data. For example, processing circuitry 80 may transmit an instruction to IMD 16 which requests IMD 16 to export collected data (e.g., a portion of collected physiological data) to computing device 300. In turn, computing device 300 may receive the collected data from IMD 16 and store the collected data in memory 84. Additionally, or alternatively, processing circuitry 80 may export instructions to IMD 16 requesting IMD 16 to update electrode combinations for stimulation or sensing according to operational parameters 90.

A user, such as a clinician or patient 4, may interact with computing device 300 through user interface 92. User interface 92 includes a display (not shown), such as an LCD or LED display or other type of screen, with which processing circuitry 80 may present information related to IMD 16 (e.g., EGM signals obtained from at least one electrode or at least one electrode combination). In addition, user interface 92 may include an input mechanism to receive input from the user. The input mechanisms may include, for example, any one or more of buttons, a keypad (e.g., an alphanumeric keypad), a peripheral pointing device, a touch screen, or another input mechanism that allows the user to navigate through user interfaces presented by processing circuitry 80 of computing device 300 and provide input. In other examples, user interface 92 also includes audio circuitry for providing audible notifications, instructions or other sounds to patient 4, receiving voice commands from patient 4, or both. Memory 84 may include instructions for operating user interface 92 and for managing power source 94.

Power source 94 is configured to deliver operating power to the components of computing device 300. Power source 94 may include a battery and a power generation circuit to produce the operating power. In some examples, the battery is rechargeable to allow extended operation. Recharging may be accomplished by electrically coupling power source 94 to a cradle or plug that is connected to an alternating current (AC) outlet. In addition, recharging may be accomplished through proximal inductive interaction between an external charger and an inductive charging coil within computing device 300. In other examples, traditional batteries (e.g., nickel cadmium or lithium ion batteries) may be used. In addition, computing device 300 may be directly coupled to an alternating current outlet to operate.

Computing device 300 may be configured to communicate with IMD 16 to receive data packets that are signed using a private key of IMD 16. That is, computing device 300 may receive data packets and one or more digital signatures associated with the data packets. In some examples, each data packet received by computing device 300 may be associated with an individual digital signature. In some examples, a group of data packets received by computing device 300 may be associated with a digital signature. As such, a digital signature received by computing device 300 from IMD 16 may be associated with one or more data packets received by computing device 300 from IMD 16.

Processing circuitry 80 of computing device 300 may be configured to validate the data integrity of one or more data packets received, via communication circuitry 82, from IMD 16 by verifying the digital signature received, via communication circuitry 82, from IMD 16 that is associated with the one or more data packets. Because the digital signature is generated based at least in part on IMD 16 signing the one or more data packets using a private key that corresponds to public key 98 included in digital certificate 96 stored in memory 84, processing circuitry 80 may be configured to verify the digital signature associated with the one or more data packets using public key 98 included in digital certificate 96 stored in memory 84.

For example, processing circuitry 80 may be configured to perform a hashing algorithm to hash the one or more packets to generate a hash. Processing circuitry 80 may also be configured to decrypt the digital signature using public key 98 to determine a public key and a first proof value from the digital signature. Processing circuitry 80 may use the public key in the decrypted digital signature to determine a second proof value from the hash. Processing circuitry 80 may therefore compare the first proof value and the second proof to validate the data integrity of the one or more data packets. For example, if processing circuitry 80 determines that the first proof value matches (e.g., is the same as) the second proof value, processing circuitry 80 may successfully validate the data integrity of the one or more data packets received from IMD 16.

In some examples, processing circuitry 80 may be configured to upgrade the firmware of the components of IMD 16, such as the firmware of communication circuitry 38 of IMD 16, by sending, via communication circuitry 82, firmware upgrade data to IMD 16. In some examples, processing circuitry 80 may be configured to send, via communication circuitry 82 to IMD 16, a firmware upgrade package that includes the firmware upgrade data.

In some examples, processing circuitry 80 may be configured to generate a code signature associated with the firmware upgrade data, and to send, via communication circuitry 82, a firmware upgrade package that includes the firmware upgrade data and the code signature to IMD 16. For example, processing circuitry 80 may be configured to sign the firmware upgrade data using a private key to generate a digital signature as the code signature associated with the firmware upgrade data.

In some examples, processing circuitry 80 may be configured to include a digital certificate in the firmware upgrade package that is sent to IMD 16. The digital certificate may specify information such as the source (e.g., computing device 300) of the firmware upgrade data, the IMD(s) to which the firmware upgrade data is applicable, and/or the patient(s) for which the firmware upgrade data is intended. By specifying such information in the digital certificate, IMD 16 may be able to verify that the firmware upgrade data is from a trusted source, that the firmware upgrade data is applicable to the specific IMD 16, and/or that the firmware upgrade data is intended for patient 4 by verifying the digital certificate.

In some examples, processing circuitry 80 may be configured to sign the digital certificate using a private key to generate a digital signature that is included in the firmware upgrade package. IMD 16 may therefore be able to verify the digital certificate in order to verify whether the firmware upgrade data is from a trusted source, whether the firmware upgrade data is applicable to the specific IMD 16, and/or whether the firmware upgrade data is intended for patient 4.

In some examples, processing circuitry 80 may be configured to upgrade a digital certificate stored in IMD 16 by sending, via communication circuitry 82, certificate upgrade data to IMD 16. The certificate upgrade data may include data for upgrading a digital certificate stored in IMD 16 or may include an upgraded digital certificate to replace a digital certificate in IMD 16. In some examples, processing circuitry 80 may be configured to send, via communication circuitry 82 to IMD 16, a certificate upgrade package that includes the certificate upgrade data.

Processing circuitry 80 may be configured to sign the certificate upgrade data using a private key that corresponds to a public key associated with a digital certificate of IMD 16 to generate a digital signature. Processing circuitry 80 may therefore be configured to include the digital signature in the certificate upgrade package that is sent to IMD 16.

In some examples, processing circuitry 80 may be configured to include a digital certificate in the certificate upgrade package that is sent to IMD 16. The digital certificate may specify information such as the source (e.g., computing device 300) of the certificate upgrade data, the IMD(s) to which the certificate upgrade data is applicable, and/or the patient(s) for which the certificate upgrade data is intended. By specifying such information in the digital certificate, IMD 16 may be able to verify that the certificate upgrade data is from a trusted source, that the certificate upgrade data is applicable to the specific IMD 16, and/or that the certificate upgrade data is intended for patient 4 by verifying the digital certificate.

In some examples, processing circuitry 80 may be configured to sign the digital certificate using a private key to generate a digital signature that is included in the certificate upgrade package. IMD 16 may therefore be able to verify the digital certificate in order to verify whether the certificate upgrade data is from a trusted source, whether the firmware upgrade data is applicable to the specific IMD 16, and/or whether the firmware upgrade data is intended for patient 4.

In some examples, if every root certificate in IMD 16 is expired, processing circuitry 80 may be configured to digitally sign the certificate upgrade data using a private key associated with an expired root certificate of IMD 16. Thus, if a private key is associated with an expired root certificate of IMD 16, processing circuitry 80 may be configured to digitally sign the certificate upgrade data using that private key.

In some examples, if every root certificate stored in IMD 16 is expired, processing circuitry 80 may be configured to send the certificate upgrade package signed using a private key associated with an expired root certificate via an out-of-band communication link to IMD 16. For example, if computing device 300 is an example of external device 20 that communicates with IMD 16 via communication link 26, computing device 300 may establish another communication link with IMD 16 separate from communication link 26, and may transmit the certificate upgrade package to IMD 16 using the communication link that is separate from communication link 26. Similarly, if computing device 300 is an example of computing system 24 that communicates with IMD 16 via communication link 28, computing device 300 may establish another communication link with IMD 16 separate from communication link 28, and may transmit the certificate upgrade package to IMD 16 using the communication link that is separate from communication link 28.

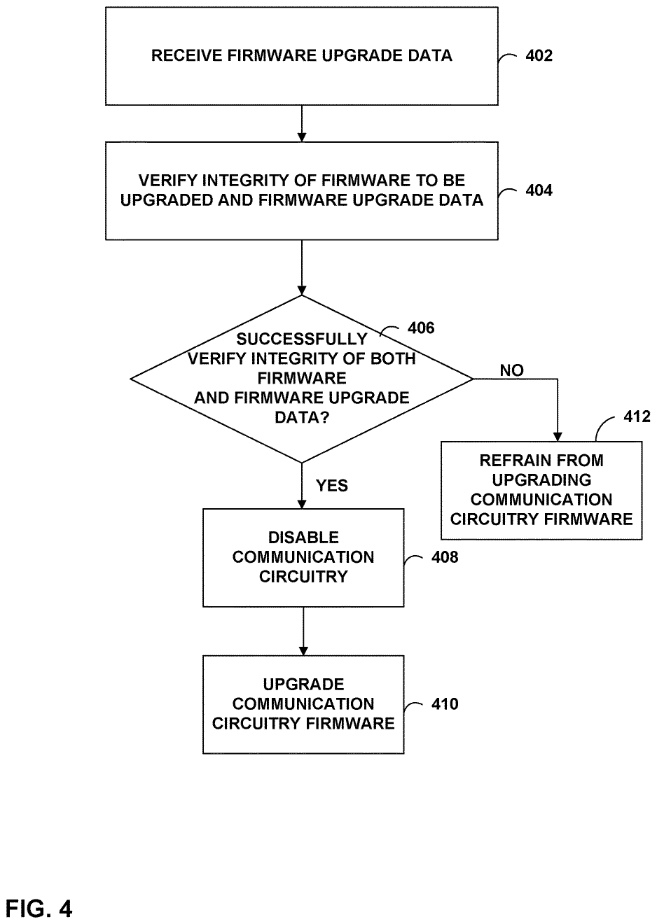

FIG. 4 is a flow diagram illustrating an example operation of an example implantable medical device to perform a firmware upgrade in accordance with one or more techniques of this disclosure. The example operation is described with respect to IMD 16 of FIGS. 1-3, and components thereof.

As shown in FIG. 4, processing circuitry 30 of IMD 16 may be configured to receive, via communication circuitry 38, firmware upgrade data for performing a firmware upgrade on communication circuitry firmware 56 (402). For example, IMD 16 may be configured to perform a firmware upgrade on communication circuitry firmware 56 of a Bluetooth radio in communication circuitry 38. Processing circuitry 30 may be configured to receive, via communication circuitry 38 and from an external device (e.g., external device 20), firmware upgrade data, such as in the form of a firmware upgrade image, and may store the firmware upgrade data in a sparse location in memory 40.

Processing circuitry 30 may verify the integrity of communication circuitry firmware 56 to be upgraded as well as the integrity of the firmware upgrade data (404). Processing circuitry 30 may verify the integrity of communication circuitry firmware 56 via any suitable technique, such as by generating a hash of communication circuitry firmware 56 and comparing the hash of communication circuitry firmware 56 against a previously-determined hash of communication circuitry firmware 56.

Processing circuitry 30 may verify the integrity of the firmware upgrade data by performing code signature verification of the firmware upgrade data. In some examples, processing circuitry 30 may receive, via communication circuitry 38, a firmware upgrade package that includes the firmware upgrade data and a code signature associated with the firmware upgrade data, and processing circuitry 30 may be configured to verify the integrity of the firmware upgrade data based at least in part on verifying the code signature associated with the firmware upgrade data. Such a code signature may be a result of external device 20 and/or computing system 24 digitally signing the firmware upgrade data using a private key that corresponds to a public key associated with a digital certificate out of digital certificates 52 stored in memory 40 to generate the code signature. Processing circuitry 30 may therefore, in some examples, use a public key that corresponds to the digital certificate out of digital certificates 52 to verify the code signature in order to verify the integrity of the firmware upgrade data.

In some examples, the firmware upgrade package may include a digital certificate associated with the firmware upgrade data that may specify information such as the source of the firmware upgrade data, the IMD(s) to which the firmware upgrade data is applicable, and/or the patient(s) for which the firmware upgrade data is intended. By verifying the digital certificate, processing circuitry 30 may be able to verify that the firmware upgrade data is from a trusted source, that the firmware upgrade data is applicable to the specific IMD 16, and/or that the firmware upgrade data is intended for patient 4.

In some examples, the digital certificate included in the firmware upgrade package is signed using a private key that corresponds to a digital certificate out of digital certificates 52 stored in memory 40 to generate the code signature. That is, the digital certificate may include or be associated with a digital signature that is a result of signing the digital certificate using such a private key. Processing circuitry 30 may therefore be configured to use a public key from a digital certificate out of digital certificates 52 that corresponds to the private key used to sign the digital certificate to verify the digital certificate in order to verify whether the firmware upgrade data is from a trusted source, whether the firmware upgrade data is applicable to the specific IMD 16, and/or whether the firmware upgrade data is intended for patient 4.

Processing circuitry 30 may determine whether processing circuitry 30 is able to successfully verify the integrity of communication circuitry firmware 56 as well as the integrity of the firmware upgrade data (406). If processing circuitry 30 is able to successfully verify the integrity of communication circuitry firmware 56 as well as the integrity of the firmware upgrade data (YES at 406), processing circuitry 30 may disable communication circuitry 38 that is to be upgraded (408) and may apply the firmware upgrade data to communication circuitry firmware 56 to upgrade communication circuitry firmware 56 (410). If processing circuitry 30 is not able to successfully verify both the integrity of communication circuitry firmware 56 and the integrity of the firmware upgrade data (NO at 406), processing circuitry 30 may refrain from upgrading the communication circuitry firmware 56 (412).

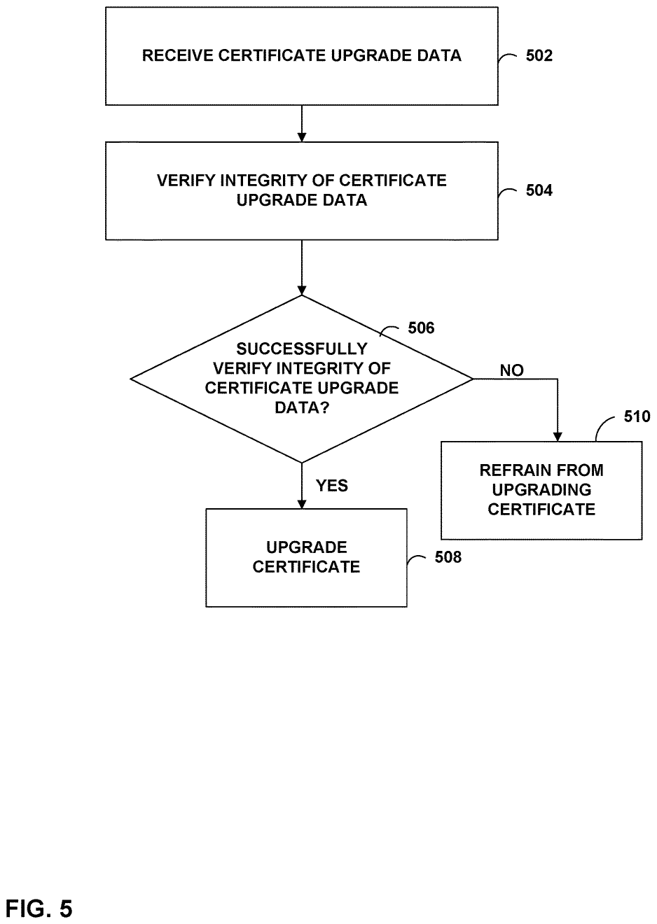

FIG. 5 is a flow diagram illustrating an example operation of an example implantable medical device to perform a certificate upgrade in accordance with one or more techniques of this disclosure. The example operation is described with respect to IMD 16 of FIGS. 1-3, and components thereof.

As shown in FIG. 5, processing circuitry 30 of IMD 16 may receive certificate upgrade data for upgrading a digital certificate stored in memory 40, such as digital certificate 52A, from external device 20 and/or computing system 24 (502). The certificate upgrade data may include an upgraded digital certificate to replace digital certificate 52A or may include code for modifying digital certificate 52A to generate an upgraded digital certificate in memory 40.

Processing circuitry 30 may verify the integrity of the certificate upgrade data based at least in part on verifying a digital signature associated with the certificate upgrade data (504). Such a digital signature may be a result of external device 20 and/or computing system 24 digitally signing the certificate upgrade data using a public key that corresponds to a public key associated with a digital certificate of digital certificates 52 to generate the digital signature. Processing circuitry 30 may therefore, in some examples, use a public key associated with a digital certificate of digital certificates 52 that corresponds to the private key used to sign the certificate upgrade data to verify the digital signature in order to verify the integrity of the certificate upgrade data.

In some examples, in addition to the digital signature or in the alternative, the certificate upgrade package received by IMD 16 may also include a digital certificate or other form of data that indicates the source of the certificate upgrade data, the intended IMD to be upgraded by the certificate upgrade data, and/or the patient having the IMD that is to be upgraded by the certificate upgrade data. Processing circuitry 30 may therefore, in some examples, verify the integrity of the certificate upgrade data based at least in part on verifying the digital certificate associated with the certificate upgrade data. If the digital certificate may include a digital signature signed using a private key that corresponds to a public key associated with a digital certificate of digital certificates 52, processing circuitry 30 may use a public key associated with a digital certificate of digital certificates 52 that corresponds to the private key used to sign the digital certificate to verify the digital signature.

Processing circuitry 30 may determine whether processing circuitry 30 is able to successfully verify the certificate upgrade data (506). If processing circuitry 30 is able to successfully verify the integrity of the certificate upgrade data (YES at 506), processing circuitry 30 may apply the certificate upgrade data to upgrade a digital certificate stored in memory 40, such as digital certificate 52A (508). If processing circuitry 30 is not able to successfully verify the integrity of the certificate upgrade data (NO at 506), processing circuitry 30 may refrain from upgrading a digital certificate stored in memory 40 (510).

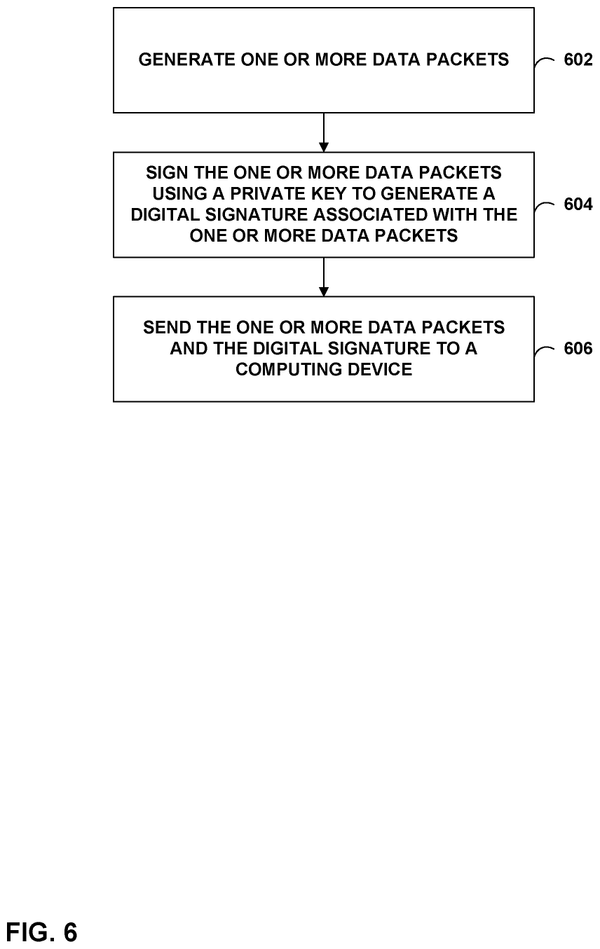

FIG. 6 is a flow diagram illustrating an example operation in accordance with one or more techniques of this disclosure. The example operation is described with respect to IMD 16 of FIGS. 1-3, and components thereof.

As shown in FIG. 6, processing circuitry 30 of medical device 16 may generate one or more data packets (602). In some examples, medical device 16 may be an implantable medical device. In some examples, the one or more data packets may include physiological data associated with a patient 4.

Processing circuitry 30 may sign the one or more data packets using a private key 54A to generate a digital signature associated with the one or more data packets (604). In some examples, a digital certificate 52A may be stored in the medical device 16. In some examples, the digital certificate 52A comprises an X.509 certificate.

In some examples, the one or more data packets include a plurality of data packets. In some examples, to sign the one or more data packets, processing circuitry 30 may send each of the plurality of data packets to generate, for each of the plurality of data packets, a respective digital signature. In some examples, to send the one or more data packets and the digital signature to the computing device 300, processing circuitry 30 may send the plurality of data packets and the respective digital signature for each of the plurality of data packets to the computing device 300.

In some examples, the one or more data packets include a first group of data packets and a second group of data packets. In some examples, to sign the one or more data packets, processing circuitry 30 may sign the first group of data packets to generate a first digital signature associated with the first group of data packets and may sign the second group of data packets to generate a second digital signature associated with the second group of data packets. In some examples, to send the one or more data packets and the digital signature to the computing device 300, processing circuitry 30 may send the first group of data packets, the first digital signature, the second group of data packets, and the second digital signature to the computing device 300.

In some examples, processing circuitry 30 may select the private key 54A for signing the one or more data packets out of a plurality of private keys 54 stored in the medical device 16 based at least in part on the computing device 300 to which the data packet is sent. In some examples, to select the private key 54A for signing the one or more data packets out of a plurality of private keys 54 stored in the medical device 16, processing circuitry 30 may select the private key 54A for signing the one or more data packets out of the plurality of private keys 54 stored in the medical device 16 based at least in part on a type of the computing device 300. In some examples, to select the private key 54A for signing the one or more data packets out of a plurality of private keys 54 stored in the medical device 16, processing circuitry 30 may select the private key 54A for signing the one or more data packets out of the plurality of private keys 54 stored in the medical device 16 based at least in part on a geographic location of the computing device 300.

Processing circuitry 30 may send the one or more data packets and the digital signature to a computing device 300 (606). In some examples, the computing device 300 is a mobile computing device, and to send the one or more data packets and the digital signature to the computing device 300, processing circuitry 30 may send the one or more data packets and the digital signature to the mobile computing device via a wireless communication link 26. In some examples, the mobile computing device may be a medical device programmer. In some examples, the mobile computing device may be a smart phone. In some examples, the wireless communication link 26 may be a Bluetooth communication link.

In some examples, the computing device 300 is a computing system 24. In some examples, the computing system 24 is a cloud-based computing system. In some examples, to send the one or more data packets and the digital signature to the computing device 300, processing circuitry 30 may send the one or more data packets and the digital signature to the computing system 24 via the Internet.

In some examples, the digital signature is able to be verified by the computing device using a public key that is part of an asymmetric key pair with the private key.

Aspects of this disclosure include the following exemplary clauses.

Clause 1. A method comprising: generating, by processing circuitry of a medical device, one or more data packets; signing, by the processing circuitry of a medical device, one or more data packets using a private key stored in the medical device to generate a digital signature associated with the one or more data packets; and sending, by the processing circuitry, the one or more data packets and the digital signature to a computing device.

Clause 2. The method of clause 1, wherein a digital certificate stored in the medical device is associated with the private key.

Clause 3. The method of clause 2, wherein the digital certificate comprises an X.509 certificate.

Clause 4. The method of any of clauses 1-3, wherein: the one or more data packets include a plurality of data packets; signing the one or more data packets further comprises signing, by the processing circuitry, each of the plurality of data packets to generate, for each of the plurality of data packets, a respective digital signature; and sending, by the processing circuitry, the one or more data packets and the digital signature to the computing device further comprises sending, by the processing circuitry, the plurality of data packets and the respective digital signature for each of the plurality of data packets to the computing device.

Clause 5. The method of any of clauses 1-3, wherein: the one or more data packets include a first group of data packets and a second group of data packets; signing the one or more data packets further comprises: signing, by the processing circuitry, the first group of data packets to generate a first digital signature associated with the first group of data packets, and signing, by the processing circuitry, the second group of data packets to generate a second digital signature associated with the second group of data packets; and sending, by the processing circuitry, the one or more data packets and the digital signature to the computing device further comprises sending, by the processing circuitry, the first group of data packets, the first digital signature, the second group of data packets, and the second digital signature to the computing device.

Clause 6. The method of any of clauses 1-5, further comprising: selecting, by the processing circuitry, the private key for signing the one or more data packets out of a plurality of private keys stored in the medical device based at least in part on the computing device to which the data packet is sent.

Clause 7. The method of clause 6, wherein selecting the private key further comprises: selecting, by the processing circuitry, the private key for signing the one or more data packets out of the plurality of private keys stored in the medical device based at least in part on a type of the computing device.

Clause 8. The method of any of clauses 6 or 7, wherein selecting the private key further comprises: selecting, by the processing circuitry, the private key for signing the one or more data packets out of the plurality of private keys stored in the medical device based at least in part on a geographic location of the computing device.

Clause 9. The method of any of clauses 1-8, wherein the computing device comprises a mobile computing device, and wherein sending the one or more data packets and the digital signature to a computing device comprises: sending, by the processing circuitry, the one or more data packets and the digital signature to the mobile computing device via a wireless communication link.

Clause 10. The method of clause 9, wherein the mobile computing device comprises a medical device programmer.

Clause 11. The method of clause 9, wherein the mobile computing device comprises a smart phone.

Clause 12. The method of any of clauses 9-11, wherein the wireless communication link comprises a Bluetooth communication link.

Clause 13. The method of any of clauses 1-8 or 12, wherein the computing device comprises a computing system.

Clause 14. The method of clause 13, wherein the computing system comprises a cloud-based computing system.

Clause 15. The method of any of clauses 13 or 14, wherein sending the one or more data packets and the digital signature to a computing device comprises: sending, by the processing circuitry, the one or more data packets and the digital signature to the computing system via the Internet.

Clause 16. The method of any of clauses 1-15, wherein the digital signature is able to be verified by the computing device using a public key that is part of an asymmetric key pair with the private key.

Clause 17. The method of any of clauses 1-16, wherein the one or more data packets include physiological data associated with a patient.

Clause 18. The method of any of clauses 1-17, wherein the medical device comprises an implantable medical device.

Clause 19. A medical device comprising: memory configured to store a private key; communication circuitry; and processing circuitry electrically coupled to the communication circuitry and the memory, wherein the processing circuitry is configured to: generate one or more data packets; sign the one or more data packets using the private key to generate a digital signature associated with the one or more data packets; and send, using the communication circuitry, the one or more data packets and the digital signature to a computing device.

Clause 20. A medical device comprising: memory; communication circuitry; and processing circuitry electrically coupled to the communication circuitry and the memory, wherein the processing circuitry is configured to perform the method of any of clauses 1-18.

Clause 21. An apparatus comprising means for performing the method of any of clauses 1-18.

Clause 22. A non-transitory computer-readable storage medium comprising program instructions that, when executed by processing circuitry of a medical device, cause the processing circuitry to perform the methods of any of clauses 1-18.

Clause 23. A medical device comprising: memory configured to store a private key; communication circuitry; and processing circuitry electrically coupled to the communication circuitry and the memory, wherein the processing circuitry is configured to: generate one or more data packets; sign the one or more data packets using the private key to generate a digital signature associated with the one or more data packets; and send, using the communication circuitry, the one or more data packets and the digital signature to a computing device.

Clause 24. The medical device of clause 23, wherein a digital certificate stored in the medical device is associated with the private key.

Clause 25. The medical device of clause 24, wherein the digital certificate comprises an X.509 certificate.

Clause 26. The medical device of any of clauses 23-25, wherein: the one or more data packets include a plurality of data packets; to sign the one or more data packets, the processing circuitry is further configured to sign each of the plurality of data packets to generate, for each of the plurality of data packets, a respective digital signature; and to send the one or more data packets and the digital signature to the computing device, the processing circuitry is further configured to send the plurality of data packets and the respective digital signature for each of the plurality of data packets to the computing device.

Clause 27. The medical device of any of clauses 23-25, wherein: the one or more data packets include a first group of data packets and a second group of data packets; to sign the one or more data packets, the processing circuitry is further configured to sign the first group of data packets to generate a first digital signature associated with the first group of data packets, and to sign the second group of data packets to generate a second digital signature associated with the second group of data packets; and to send the one or more data packets and the digital signature to the computing device, the processing circuitry is further configured to send the first group of data packets, the first digital signature, the second group of data packets, and the second digital signature to the computing device.

Clause 28. The medical device of any of clauses 23-27, wherein the processing circuitry is further configured to: selecting, by the processing circuitry, the private key for signing the one or more data packets out of a plurality of private keys stored in the medical device based at least in part on the computing device to which the one or more data packets are sent.

Clause 29. The medical device of clause 28, wherein to select the private key, the processing circuitry is further configured to: select the private key for signing the one or more data packets out of the plurality of private keys stored in the medical device based at least in part on a type of the computing device.

Clause 30. The medical device of clause 28, wherein to select the private key, the processing circuitry is further configured to: select the private key for signing the one or more data packets out of the plurality of private keys stored in the medical device based at least in part on a geographic location of the computing device.

Clause 31. The medical device of any of clauses 23-30, wherein the computing device comprises a mobile computing device, and wherein to send the one or more data packets and the digital signature to the computing device, the processing circuitry is further configured to: send the one or more data packets and the digital signature to the mobile computing device via a wireless communication link.

Clause 32. The medical device of any of clauses 23-30, wherein the computing device comprises a computing system, and wherein to send the one or more data packets and the digital signature to the computing device, the processing circuitry is further configured to: send the one or more data packets and the digital signature to the computing system via the Internet.

Clause 33. The medical device of any of clauses 23-32, wherein the digital signature is able to be verified by the computing device using a public key that is part of an asymmetric key pair with the private key.

Clause 34. The medical device of any of clauses 23-33, wherein the one or more data packets include physiological data associated with a patient.

Clause 35. The medical device of any of clauses 23-34, wherein the medical device comprises an implantable medical device.

Clause 36. A system comprising: a medical device configured to: generate one or more data packets; sign the one or more data packets using a private key to generate a digital signature associated with the one or more data packets; and send the one or more data packets and the digital signature to a computing device; and the computing device configured to: receive the one or more data packets and the digital signature from the medical device; and verify, using a public key that corresponds to the private key, the digital signature.

Clause 37. The system of clause 36, wherein a first digital certificate stored in the medical device is associated with the private key, an wherein a second digital certificate stored in the computing device is associated with the public key.

Clause 38. The system of clause 37, wherein the first digital certificate and the second digital certificate each comprises an X.509 certificate.

Clause 39. The system of any of clauses 36-38, wherein to verify the digital signature, the computing device is further configured to: hash the one or more data packets to generate a hash value; decrypt, using the public key, the digital signature to decrypt a first proof value and a second public key; determine, from the hash value and using the second public key, a second proof value; compare the first proof value with the second proof value to verify the digital signature.

Clause 40. The system of any of clauses 36-39, wherein: the one or more data packets include a plurality of data packets; to sign the one or more data packets, the medical device is further configured to sign each of the plurality of data packets to generate, for each of the plurality of data packets, a respective digital signature; and to send the one or more data packets and the digital signature to the computing device, the medical device is further configured to send the plurality of data packets and the respective digital signature for each of the plurality of data packets to the computing device.

Clause 41. The system of any of clauses 36-39, wherein: the one or more data packets include a first group of data packets and a second group of data packets; to sign the one or more data packets, the medical device is further configured to sign the first group of data packets to generate a first digital signature associated with the first group of data packets, and to sign the second group of data packets to generate a second digital signature associated with the second group of data packets; and to send the one or more data packets and the digital signature to the computing device, the medical device is further configured to send the first group of data packets, the first digital signature, the second group of data packets, and the second digital signature to the computing device.

Clause 42. The system of any of clauses 36-41, wherein the medical device is further configured to: selecting, by the medical device, the private key for signing the one or more data packets out of a plurality of private keys stored in the medical device based at least in part on the computing device to which the one or more data packets are sent.

Clause 43. The system of clause 42, wherein to select the private key, the medical device is further configured to: select the private key for signing the one or more data packets out of the plurality of private keys stored in the medical device based at least in part on a type of the computing device.

Clause 44. The system of clause 42, wherein to select the private key, the medical device is further configured to: select the private key for signing the one or more data packets out of the plurality of private keys stored in the medical device based at least in part on a geographic location of the computing device.

Clause 45. The system of any of clauses 36-44, wherein the computing device comprises a mobile computing device, and wherein to send the one or more data packets and the digital signature to the computing device, the medical device is further configured to: send the one or more data packets and the digital signature to the mobile computing device via a wireless communication link.

Clause 46. The system of any of clauses 36-44, wherein the computing device comprises a computing system, and wherein to send the one or more data packets and the digital signature to the computing device, the medical device is further configured to: send the one or more data packets and the digital signature to the computing system via the Internet.

Clause 47. The system of any of clauses 36-46, wherein the one or more data packets include physiological data associated with a patient.

Clause 48. The system of any of clauses 36-47, wherein the medical device comprises an implantable medical device.

The techniques described in this disclosure may be implemented, at least in part, in hardware, software, firmware, or any combination thereof. For example, various aspects of the techniques may be implemented within one or more microprocessors, DSPs, ASICs, FPGAs, or any other equivalent integrated or discrete logic QRS circuitry, as well as any combinations of such components, embodied in external devices, such as physician or patient programmers, stimulators, or other devices. The terms “processor” and “processing circuitry” may generally refer to any of the foregoing logic circuitry, alone or in combination with other logic circuitry, or any other equivalent circuitry, and alone or in combination with other digital or analog circuitry.

For aspects implemented in software, at least some of the functionality ascribed to the systems and devices described in this disclosure may be embodied as instructions on a computer-readable storage medium such as RAM, DRAM, SRAM, magnetic discs, optical discs, flash memories, or forms of EPROM or EEPROM. The instructions may be executed to support one or more aspects of the functionality described in this disclosure.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements. The techniques of this disclosure may be implemented in a wide variety of devices or apparatuses, including an IMD, an external programmer, a combination of an IMD and external programmer, an integrated circuit (IC) or a set of ICs, and/or discrete electrical circuitry, residing in an IMD and/or external programmer.

1-15. (canceled)

16. A medical device comprising:
memory configured to store a private key;
communication circuitry; and
processing circuitry electrically coupled to the communication circuitry and the memory, wherein the processing circuitry is configured to:
generate one or more data packets;
sign the one or more data packets using the private key to generate a digital signature associated with the one or more data packets; and
send, using the communication circuitry, the one or more data packets and the digital signature to a computing device.

17. The medical device of claim 16, wherein:
the one or more data packets include a plurality of data packets;
to sign the one or more data packets, the processing circuitry is further configured to sign each of the plurality of data packets to generate, for each of the plurality of data packets, a respective digital signature; and
to send the one or more data packets and the digital signature to the computing device, the processing circuitry is further configured to send the plurality of data packets and the respective digital signature for each of the plurality of data packets to the computing device.

18. The medical device of claim 16, wherein:
the one or more data packets include a first group of data packets and a second group of data packets;
to sign the one or more data packets, the processing circuitry is further configured to sign the first group of data packets to generate a first digital signature associated with the first group of data packets, and to sign the second group of data packets to generate a second digital signature associated with the second group of data packets; and
to send the one or more data packets and the digital signature to the computing device, the processing circuitry is further configured to send the first group of data packets, the first digital signature, the second group of data packets, and the second digital signature to the computing device.

19. The medical device of claim 16, wherein the processing circuitry is further configured to:
selecting, by the processing circuitry, the private key for signing the one or more data packets out of a plurality of private keys stored in the medical device based at least in part on the computing device to which the one or more data packets are sent.

20. The medical device of claim 19, wherein to select the private key, the processing circuitry is further configured to: select the private key for signing the one or more data packets out of the plurality of private keys stored in the medical device based at least in part on a type of the computing device.

21. The medical device of claim 19, wherein to select the private key, the processing circuitry is further configured to: select the private key for signing the one or more data packets out of the plurality of private keys stored in the medical device based at least in part on a geographic location of the computing device.

22. The medical of claim 16, wherein the computing device comprises a mobile computing device, and wherein to send the one or more data packets and the digital signature to the computing device, the processing circuitry is further configured to:
send the one or more data packets and the digital signature to the mobile computing device via a wireless communication link.

23. The medical device of claim 16, wherein the computing device comprises a computing system, and wherein to send the one or more data packets and the digital signature to the computing device, the processing circuitry is further configured to:
send the one or more data packets and the digital signature to the computing system via the Internet.

24. The medical device of claim 16, wherein the digital signature is able to be verified by the computing device using a public key that is part of an asymmetric key pair with the private key.

25. The medical device of claim 16, wherein the one or more data packets include physiological data associated with a patient.

26. The medical of claim 16, wherein the medical device comprises an implantable medical device.

27. A method comprising:
generating, by processing circuitry of a medical device, one or more data packets;
signing, by the processing circuitry of a medical device, one or more data packets using a private key stored in the medical device to generate a digital signature associated with the one or more data packets; and
sending, by the processing circuitry, the one or more data packets and the digital signature to a computing device.

28. The method of claim 27,
wherein the one or more data packets include a plurality of data packets;
wherein signing the one or more data packets further comprises signing, by the processing circuitry, each of the plurality of data packets to generate, for each of the plurality of data packets, a respective digital signature; and
wherein sending, by the processing circuitry, the one or more data packets and the digital signature to the computing device further comprises sending, by the processing circuitry, the plurality of data packets and the respective digital signature for each of the plurality of data packets to the computing device.

29. The method of claim 27,
wherein the one or more data packets include a first group of data packets and a second group of data packets;
wherein signing the one or more data packets further comprises: signing, by the processing circuitry, the first group of data packets to generate a first digital signature associated with the first group of data packets, and signing, by the processing circuitry, the second group of data packets to generate a second digital signature associated with the second group of data packets; and
wherein sending, by the processing circuitry, the one or more data packets and the digital signature to the computing device further comprises sending, by the processing circuitry, the first group of data packets, the first digital signature, the second group of data packets, and the second digital signature to the computing device.

30. The method of claim 27, further comprising:
selecting, by the processing circuitry, the private key for signing the one or more data packets out of a plurality of private keys stored in the medical device based at least in part on the computing device to which the data packet is sent.

31. The method of claim 30, wherein selecting the private key further comprises:
selecting, by the processing circuitry, the private key for signing the one or more data packets out of the plurality of private keys stored in the medical device based at least in part on a type of the computing device.

32. The method of claim 30, wherein selecting the private key further comprises: selecting, by the processing circuitry, the private key for signing the one or more data packets out of the plurality of private keys stored in the medical device based at least in part on a geographic location of the computing device.

33. The method of claim 27, wherein the computing device comprises a mobile computing device, and wherein sending the one or more data packets and the digital signature to a computing device comprises:
sending, by the processing circuitry, the one or more data packets and the digital signature to the mobile computing device via a wireless communication link.

34. The method of claim 27, wherein the digital signature is able to be verified by the computing device using a public key that is part of an asymmetric key pair with the private key.

35. A non-transitory computer-readable storage medium comprising program instructions that, when executed by processing circuitry of a medical device, cause the processing circuitry to:
generate one or more data packets;
sign the one or more data packets using a private key to generate a digital signature associated with the one or more data packets; and
send, using the communication circuitry, the one or more data packets and the digital signature to a computing device.
